(19) United States
(12) Patent Application Publication
(10) Pub. No.: US 2009/0239213 A1
(43) Pub. Date: Sep. 24, 2009

(54) Identifying a target polynucleotide

(75) Inventor: Preben Lexow, Oslo (NO)

Correspondence Address: MARSHALL, GERSTEIN & BORUN LLP, 233 SOUTH WACKER DRIVE, 6300 SEARS TOWER, CHICAGO, IL, 60606-6357, US

(73) Assignee: LINGVITAE AS, Oslo (NO)

(21) Appl. No.: 11/577,028

(22) PCT Filed: Oct. 12, 2005
(86) PCT No.: PCT/GB05/03923

§ 371 Date: Apr. 2, 2008

(30) Foreign Application Priority Data
Oct. 13, 2004 (GB) 0422730.2

(51) Int. Cl. C12Q 1/68

(52) U.S. Cl. 435/6

(57) ABSTRACT

A method for identifying the presence of a single stranded target polynucleotide in a sample comprising the steps of (i) contacting the target polynucleotide, under hybridising conditions, with at least first and second polynucleotide probes, each of which comprise a first region complementary to adjacent non-overlappïng regions of the target polynucleotide; (ii) ligating together those first and second polynucleotides that hybridise to the target polynucleotide; (iii) optionally, amplifying any ligated polynucleotides; and (iv) determining whether the target polynucleotide is present in the original sample, by detecting any ligated polynucleotide, wherein at least one of the first and second polynucleotides comprise a second region having a defined polynucleotide sequence, with each individual nucleotide of the first region being represented by at least two nucleotides on the second region, and the ligated polynucleotide being identified by determining the second region of at least one of the first and second polynucleotide probes.

### FIELD OF THE INVENTION

This invention relates to methods for detecting a target polynucleotide and determining its sequence, in particular characterising mutations present in the target polynucleotide.

### BACKGROUND TO THE INVENTION

It is now recognised that many disorders are attributable to mutations present in a subject's genome. Even if the symptoms of a disorder are not apparent, the presence of a specific mutation may indicate an increased risk of developing the disorder, at a future date. There is therefore much research being carried out identifying mutations that are associated with specific diseases, and developing diagnostic tests to help predict the likelihood of developing the disease.

Although many disease-related mutations have been characterised, there is still a need to produce an efficient and reliable method for determining the presence of specific target sequences, especially those containing mutations, in a particular subject. Typically, a mutation will be identified by sequencing genomic DNA obtained from a patient, and comparing the sequence information with a control.

The principal method in general use for large-scale DNA sequencing is the chain termination method. This method was first developed by Sanger and Coulson (Sanger et al., Proc. NatI. Acad. Sci. USA, 1977; 74: 5463-5467), and relies on the use of dideoxy derivatives of the four nucleotides which are incorporated into the nascent polynucleotide chain in a polymerase reaction. Upon incorporation, the dideoxy derivatives terminate the polymerase reaction and the products are then separated by gel electrophoresis and analysed to reveal the position at which the particular dideoxy derivative was incorporated into the chain.

Although this method is widely used and produces reliable results, it is recognised that it is slow, labour-intensive and expensive.

U.S. Pat. No. 5,302,509 discloses a method to sequence a polynucleotide immobilised on a solid support. The method relies on the incorporation of 3-blocked bases A, G, C and T having a different fluorescent label to the immobilised polynucleotide, in the presence of DNA polymerase. The polymerase incorporates a base complementary to the target polynucleotide, but is prevented from further addition by the 3′-blocking group. The label of the incorporated base can then be determined and the blocking group removed by chemical cleavage to allow further polymerisation to occur. However, the need to remove the blocking groups in this manner is time-consuming and must be performed with high efficiency.

A further difficulty with many existing techniques is that they do not allow the characterisation of multiple mutations which may be present in a single genome. It is therefore useful to have a method that permits the characterisation of multiple mutations in a single assay procedure, and that can detect any mutation, from a single point mutation to a gross chromosomal rearrangement.

### SUMMARY OF THE INVENTION

The present invention is based on the realisation that the presence of a target polynucleotide molecule in a sample can be detected by hybridising to the target polynucleotide at least two polynucleotide probes, at least one of which encodes information on the target sequence, linking the probes to form a single information-containing polynucleotide and detecting the linked product. The sequence of the target polynucleotide can be determined by decoding the information in the linked polynucleotide.

According to a first aspect of the invention, a method for identifying the presence of a single-stranded target polynucleotide in a sample comprises the steps of:

(i) contacting the target polynucleotide, under hybridising conditions, with at least first and second polynucleotide probes, each of which comprise a first region complementary to adjacent non-overlapping regions of the target polynucleotide;

(ii) ligating together those first and second polynucleotides that hybridise to the target polynucleotide;

(iii) optionally, amplifying any ligated polynucleotides; and

(iv) determining whether the target polynucleotide is present in the original sample, by detecting any ligated polynucleotide, wherein at least one of the first and second polynucleotides comprise a second region having a defined polynucleotide sequence, with each individual nucleotide of the first region being represented by at least two nucleotides on the second region, and the ligated polynucleotide being identified by determining the second region of at least one of the first and second polynucleotide probes.

A second aspect of the invention allows the order of sequences on a target polynucleotide to be determined. According to this aspect, a method for identifying the order of at least two target sequences on a target polynucleotide comprises the steps of:

(i) contacting the target polynucleotide, under hybridising conditions, with at least a first and second polynucleotide probe, each of which comprise a first region complementary to non-adjacent regions of the target polynucleotide;

(ii) performing a polymerase reaction to incorporate nucleotides between the at least two probes to thereby form a third polynucleotide; and

(iii) determining the order of, and distance between, the two target sequences on the target polynucleotide, by detecting any third polynucleotide, wherein the first and second polynucleotides comprise a second region having a defined polynucleotide sequence, with each individual nucleotide of the first region being represented by at least two nucleotides on the second region, and the order of and distance between the probes in the third polynucleotide being identified by detecting the second region of the first and second polynucleotide probes.

The present invention enables the detection of multiple target molecules, and the characterisation of multiple specific mutations, in a single reaction. Any mutation, from a single point mutation to a gross chromosomal rearrangement, can be characterised. The information on each mutation is encoded within a molecule which can be isolated and characterised at the end of the analysis procedure.

### DETAILED DESCRIPTION OF THE INVENTION

The present invention is used to detect a target polynucleotide and characterise mutations within the target polynucleotide. The target polynucleotide is contacted with at least two polynucleotide probes that can hybridise to separate regions of the target polynucleotide. The hybridised probes are covalently linked to form a single polynucleotide that contains information on the target polynucleotide, which is then detected and characterised. If the target sequence is not present in the sample, no hybridisation will occur and the probes will not be detected.

A large number of polynucleotide probes, designed to hybridise to a number of different target sequences, may be added to a sample simultaneously or sequentially. Preferably, the sample is a genomic DNA sample. Performing the method of the invention will identify whether the sample contains any of the target sequences that complement the probes; for each target sequence that two or more probes hybridise to, a linked polynucleotide containing at least two probes will be produced. This method allows the characterisation of multiple mutations in a single assay procedure.

The term “polynucleotide” is well known in the art and is used to refer to a series of linked nucleic acid molecules, e.g. DNA or RNA. Nucleic acid mimics, e.g. PNA, LNA (locked nucleic acid) and 2′-O-methRNA are also within the scope of the invention. It will be apparent to the skilled person that the most usual source of the target polynucleotide will be genomic DNA, obtained from a subject. In order for oligonucleotide probes to hybridise to the target polynucleotide, both the probes and target must be single-stranded at the time of hybridisation.

As used herein, the term “base” refers to each nucleic acid monomer, A, T (U), G or C. These abbreviations represent the nucleotide base adenine, thymine (uracil) guanine and cytosine. Uracil replaces thymine when the polynucleotide is RNA, or it can be introduced into DNA using dUTP, again as well understood in the art.

As used herein, the term “mutant” refers to a sequence that differs to a control sequence by at least one nucleotide. The mutation may be a substitution, deletion or insertion of one or more specific nucleic acid bases. Preferably, the present invention will be used to identify one or more single nucleotide polymorphisms (SNPs) present on a genomic DNA fragment. The term “polymorphism” as used herein refers to the occurrence of two or more alternative genomic sequences or alleles between or among different genomes or subjects. A SNP is a single base change. Typically, a SNP is the replacement of one nucleotide by another nucleotide at the polymorphic site. Deletion of a single nucleotide, or insertion of a single nucleotide, also gives rise to single nucleotide polymorphisms.

In an alternative preferred embodiment, mutations that span greater than one base are detected.

The invention requires polynucleotide probes that can hybridise to separate, non-overlapping regions of the target polynucleotide. Each probe may be the same size, or each probe may differ in size. Preferably, the regions of the target polynucleotide to which the probes hybridise are between 2 and 50 bases long, more preferably between 3 and 20 bases.

At least two probes hybridise to the target polynucleotide. Preferably, between 2 and 10 probes hybridise, most preferably 2, 3 or 4. Once hybridised, the probes can be linked together to form a single polynucleotide that contains information on the sequences of the target polynucleotide to which the probes were hybridised.

Each probe may be complementary to identical target sequences, i.e. a plurality of a single probe is added to the target sequence, or probes may be added that are complementary to different target sequences. As will be appreciated by one skilled in the art, the use of two or more identical probes will allow the identification of sequence repeats in the target polynucleotide. Triplet repeats are linked to several genetic disorders, for example fragile X syndrome is associated with a CGG triplet repeat, Myotonic dystrophy is associated with a CTG repeat and Huntingdon's Disease is associated with a CAG repeat. These diseases typically involve an increase in the number of repeat sequences in an affected individual. It is therefore possible to design probes that hybridise to the repeat sequences. The probe may hybridise to a single repeat, i.e. 3 bases in the case of triplet repeats, or may hybridise to a multiple of repeats. Hybridisation of the probes, and subsequent characteristics of the single polynucleotide formed by probe linkage, will indicate the number of repeat present and is therefore useful in the diagnoses of disorders associated with an alteration of repeat sequences.

The probes may alternatively complement different target sequences. These may span the region about a single mutation or may each be complementary to a different mutation, allowing the identification of multiple mutations from a single linked polynucleotide. If the position of a mutation in a target polynucleotide is known or suspected, but the specific mutation is not, a number of probes may be added that span the putative mutation site and contain different sequences. For example, for a particular SNP, four different probes can be used, each containing a different base at the putative mutation site. Only probes that are complementary to the target will hybridise and be ligated. Detection of the ligated polynucleotide will therefore identify the sequence at the putative mutation site.

It is preferred that the nucleotide or nucleotides which are suspected of mutation in the target polynucleotide are complementary to a terminal base, or bases, in the probe. For example, a probe may hybridise to a region containing a SNP, with the terminal base in the probe hybridising to the nucleotide position in the target that is altered by the SNP. More preferably, the terminal base in the probe that binds to the SNP site is to be covalently linked to another probe. This ensures maximum specificity of the probes, as ligation between probes will only occur if the terminal base of each probe, that is to be ligated, is hybridised to the target polynucleotide. If the mutation spans greater than one base in the target polynucleotide, the complementary bases may be contained within a single probe or spread across 2 or more probes. For example, if a mutation involves two adjacent bases in the target, the probes can be designed so that the complementary bases are either both at the terminus of one probe, or alternatively, so that the first mutated base is complementary to the last base in upstream probe, and the second mutated base is complementary to the first base in downstream probe.

The region of each probe that is complementary to the target polynucleotide is referred to herein as the “first region”. By “complementary”, it is meant that they hybridise to the target under stringent hybridising conditions. Conditions for stringent hybridisation will be apparent to one skilled in the art, an example of such conditions is overnight incubation at 42° C. in a solution comprising 50% formamide, 5×SSC (150 mM NaCl, 15 mM trisodium citrate), 50 mM sodium phosphate (pH 7.6), 5×Denhardt's solution, 10% dextran sulphate, and 20 μg/ml denatured, sheared salmon sperm DNA, followed by washing in 0.1×SSC at about 65° C. It will be apparent that the first regions of the probes do not have to be totally complementary to the target sequences, provided that they are sufficiently complementary to hybridise. However, the terminal nucleotides of each probe, that will be involved in a ligation reaction, must be complementary to the target polynucleotide in order for ligation to occur between the probe and another probe or other polynucleotide that is also hybridised to the target polynucleotide. It is most preferred that the first region of each probe is 100% complementary to a region in the target polynucleotide.

In addition to the first region of the probe that hybridises to the target polynucleotide, at least one probe that hybridises to the target also contains a second region that encodes information on the first region and therefore the sequence to which the probe hybridises. It is this second region that encodes information on the target polynucleotide. When characterising the linked polynucleotide containing the probes, it is the second region, or series of second regions, that indicate the sequence of the target polynucleotide. Each linked polynucleotide must therefore contain at least one probe second region.

The second region has a defined polynucleotide sequence, with each individual nucleotide of the first region being represented by at least two nucleotides in the second region. It is preferable that the second region comprises distinct “units” of nucleic acid sequence. Each nucleotide in the first region is represented by a distinct and pre-defined unit, or unique combination of units in the second region. Each unit will preferably comprise two, or more nucleotide bases, preferably from 2 to 50 bases, more preferably 2 to 20 bases and most preferably 4 to 10 bases, e.g. 6 bases. Preferably, there are at least two different bases contained in each unit. In a preferred embodiment, there are 3 different bases in each unit. The units in the second region may be designed according to the disclosure in WO-A-00/39333, the content of which is hereby incorporated by reference.

The design of the units is such that it will be possible to distinguish the different units during a “read-out” step, involving either the incorporation of detectably labelled nucleotides in a polymerisation reaction, or on hybridisation of complementary oligonucleotides. For example, each base in the first region is represented by a series of bases in a unit, where one base will be complementary to a labelled nucleotide introduced during the read-out step, one base will act as a “spacer” to provide separation between incorporated labels, and one base will act as a stop signal.

In a preferred embodiment, two units of distinct sequence are used to represent all of the four possible bases on the first region of the probe. According to this embodiment, the two units can be used as a binary system, with one unit representing “0” and the other representing “1”. Each base in the first region of the probe is characterised by a combination of the two units in the second region. For example, adenine may be represented by “0”+“0”, cytosine by “0”+“1”, guanine by “1”+“0” and thymine by “1”+“1”, as shown in FIG. 1. It is necessary to distinguish between the units, and so a “stop” signal can be incorporated into each unit. It is also preferable to use different units representing “1” and “0”, depending on whether the base in the first region is in an odd or even numbered position.

This is demonstrated as follows:

Odd numbered template sequence:
“0”: TTTTTTA(CCC)
“1”: TTTTTTG(CCC)
Even numbered template sequence:
“0”: CCCCCCA(TTT)
“1”: CCCCCCG(TTT)

In this example, the underlined base is the target for labelled nucleotides in a polymerase reaction, the bases in parentheses are used as a stop signal, and the remaining bases are to provide separation between the labels.

In odd numbered positions (1, 3, 5, etc) the nucleotide mix, introduced during the polymerase reaction, consists of Fluor X-dUTP, Fluor Y-dCTP and dATP (dGTP is missing from the mix). The complementary base for Fluor Y is missing for “0”, and the complementary base for Fluor X is missing for “1”. Accordingly, during a polymerase reaction, if the unit “0” is present, it will be possible to detect this by monitoring for Fluor X, and if “1” is present, by monitoring for Fluor Y.

In all even numbered positions (2, 4, 6, etc) the nucleotide mix consists of the same two fluor-labelled nucleotides, but dGTP is used, not dATP, and one or more T bases define the stop signal.

After each unit has been “read” it is possible to restart the process by introducing the missing complementary nucleotide (e.g. either dGTP or dATP) to allow incorporation at the stop sequence. Non-incorporated nucleotides are washed away prior to the next read-out step.

The first region of each polynucleotide may be converted into the second region of the polynucleotide probe using methods known in the art. Preferably, this conversion is carried out prior to the use of the probes in the current invention, so that the probes contain the second region when added to the target. The conversion method disclosed in WO-A-00/39333 (the content of which is incorporated herein by reference), using restriction enzymes, may be adopted. For example, the first region may be ligated into a vector which carries a class IIS restriction site close to the point of insertion, or the first region may be engineered to contain such a site. The appropriate class IIS restriction enzyme is then used to cleave the restriction site, resulting in an overhang in the first region.

Appropriate adapters which contain one or more of the units may then be used to bind to one or more of the bases of the overhang. Once the overhang of the adapter and the cleaved vector have been hybridised, these molecules may be ligated. This will only be achieved where full complementarity along the full extent of the overhang is achieved. Blunt-end ligation may then be effected to join the other end of the adapter to the vector. By appropriate placement of a further class II restriction site (or other appropriate restriction enzyme site), which may be same or different to the previously used enzyme, cleavage may be effected such that an overhang is created in the target sequence downstream of the sequence to which the first adapter was directed. In this way, adjacent or overlapping sequences may be consecutively converted into sequences carrying the units of defined sequence.

Using this conversion system, the second region of each polynucleotide probe is preferably formed using the binary system, wherein two consecutive units are used to define a particular base in the first region.

Once at least two probes are hybridised to the target polynucleotide, they are ligated together. The probes may be directly ligated according to the first aspect of the invention or indirectly ligated via an intervening polynucleotide according to the second aspect of the invention. (See below for further description of each aspect). Conditions suitable for ligation will be apparent to one skilled in the art; preferred conditions are the addition of a ligase enzyme under conditions suitable for ligase activity. When two probes are ligated, it is preferred that the two first regions are ligated together. The linked polynucleotide will then contain the two first regions, flanked by the two second regions. In this embodiment, the second regions of the probes are at opposite ends of the first and second probe, i.e. at the 3′ end of the first probe and the 5′ end of the second probe.

The single polynucleotide containing the covalently linked probes may optionally be amplified. A preferred method of amplification is the polymerase reaction. Conditions suitable for performing a polymerase reaction will be apparent to one skilled in the art. Typical conditions are the addition of a polymerase enzyme, an oligonucleotide primer and free nucleotides to the target template) sequence (i.e. the ligated probe molecule), under conditions suitable for polymerase activity. In a preferred embodiment, the polymerase reaction is quantitative. Quantitative polymerase reactions are well known in the art, and allow the amount of amplified product to be monitored after each cycle. In summary, the incorporation of a fluorescent signal into the amplified product is monitored, a greater amount of amplified product gives a greater fluorescent signal. Kits for quantitative, real-time PCR are commercially available, commonly used examples of which include TaqMan (Applied Biosystems Inc.) and SYBR Green (Molecular Probes Inc.).

Detection of the single molecule containing the linked probes is carried out by detection of the second regions of the probes in the “read-out” step, in which the information represented by the second region of each probe, i.e. the sequence of the target polynucleotide to which the probe hybridised, is determined. If the probes hybridised to a mutant target sequence, the mutations present on the target polynucleotide will be revealed. The read-out step may be performed using any suitable technique. Preferred embodiments are described in WO 00/39333.

A conventional sequencing procedure could be used as a read-out step to identify the second regions of the probes and thereby any mutations. Alternatively, the second regions may be detectably-labelled in a way that discriminates between different probes. The labels may be detected in the read-out step to identify the design polymers. A preferred label is a fluorophore which can be attached to the second region of each probe using conventional techniques.

The read-out phase may be achieved as discussed above using the polymerase reaction to incorporate bases complementary to those on the second region of each probe, using either selected, detectably-labelled nucleotides or nucleotides that incorporate a group for subsequent indirect labelling, and monitoring any incorporation event.

The read-out polymerase reaction is preferably carried out under conditions that permit the controlled incorporation of complementary nucleotides one unit at a time. This enables each unit to be categorised by the detection of an incorporated label. As each unit preferably comprises a “stop” sequence, it is possible to control incorporation by supplying only those nucleotides required for incorporation onto the first unit, as described above. As each unit is recognised by a specific label, it is possible to distinguish between two different units (0 and 1) within each cycle. This enables detection of any incorporated label, and allows the identification and position of the unit to be determined.

The method may be carried out as follows:
(i) contacting the second region of the probe comprising the defined units with at least one of the nucleotides dATP, dTTP, dGTP and dCTP, under conditions that permit the polymerisation reaction to proceed, wherein the at least one nucleotide comprises a detectable label specific for that nucleotide;
(ii) removing any non-incorporated nucleotides and detecting any incorporation events;
(iii) removing the labels from incorporated nucleotide; and
(iv) repeating steps ii) to iv), to thereby identify the different units, and thereby the sequence of the target polynucleotide.

The number of different nucleotides required in step (i) of each cycle will be dependent on the design of the units. If each unit comprises only one base type, then only one nucleotide (detectably labelled) is required. However, if two bases are utilised (one as a target for the detectably labelled nucleotide and one to provide a gap between different target bases) then two nucleotides will be required (one to bind to the target base and one to “fill in” the bases between the target bases).

The use of a base as a stop signal allows the detection steps to be performed without the requirement for blocked nucleotides to prevent uncontrolled incorporation during the polymerase reaction. The stop-signal is effective as the complement for the “stop” base is absent from the polymerase mix. Therefore, each unit can be characterised before a “fill-in” step is performed, using the missing nucleotide, to incorporate a complement to the stop base, which allows the next unit to be characterised. This is carried out after the detection step. The “stop” base of one unit will not be of the same type as the first base of the subsequent unit. This ensures that the “fill-in” procedure does not progress to the next unit. Non-incorporated nucleotides used in the “fill-in” procedure can then be removed, and the next unit can then be characterised.

The choice of polymerase and detectable label will be apparent to the skilled person. The following is used as a guide only:

a) Klenow and Klenow (exo-) can efficiently incorporate Tetramethylrhodamine-4-dUTP and Rhodamin-110-dCTP (Amersham Pharmacia Biotech) (Brakmann and Nieckchen, 2001, Brakmann and Löbermann, 2000).
b) Vent, Taq and Tgo DNA polymerase can efficiently incorporate dioxigenin and fluorophores like AMCA, Tetramethylrhodamin, fluorescein and Cy5 without spacing at least up to a few positions (Augustin et al., J. Biotechnol. 2001 Apr. 13; 86(3): 289-301).
c) T4 DNA polymerase is efficient in filling-in fluorophore labelled nucleotides.

The preferred polymerases are Klenow Large fragment (exo-) and T4 DNA polymerase.

To carry out the polymerase reaction it will usually be necessary to first anneal a primer sequence to the linked polynucleotide, the primer sequence being recognised by the polymerase enzyme and acting as an initiation site for the subsequent extension of the complementary strand. The primer sequence may be added as a separate component with respect to the linked polynucleotide, which comprises a complementary sequence that allows the primer to anneal.

Other conditions necessary for carrying out the polymerase reaction, including temperature, pH, buffer compositions etc., will be apparent to those skilled in the art. The polymerisation step is likely to proceed for a time sufficient to allow incorporation of bases to the first unit. Non-incorporated nucleotides are then removed, for example, by subjecting the array to a washing step, and detection of the incorporated labels may then be carried out.

An alternative read-out strategy is to use short detectably labelled oligonucleotides to hybridise to the units on the polynucleotide, and to detect any hybridisation event.

The short oligonucleotides have a sequence complementary to specific units of the second regions of the probes. For example, if a binary system is used and each characteristic is defined by a different combination of units (one representing “0” and one representing “1”) the invention will require an oligonucleotide specific for the “1” unit. In this embodiment, selective hybridisation of oligonucleotides can be achieved by designing each unit to be of a different polynucleotide sequence with respect to other units. This ensures that a hybridisation event will only occur if the specific unit is present, and the detection of hybridisation events identifies the characteristics on the target molecule.

In a preferred embodiment, the label is a fluorescent moiety. Many examples of fluorophores that may be used are known in the prior art, and include:

Alexa dyes (Molecular Probes)
BODIPY dyes (Molecular Probes)
Cyanine dyes (Amersham Biosciences Ltd.)

### Tetramethylrhodamine (Perkin Elmer, Molecular Probes, Roche Diagnostics)

### Coumarin (Perkin Elmer)

### Texas Red (Molecular Probes)

### Fluorescein (Perkin Elmer, Molecular Probes, Roche Diagnostics)

The attachment of a suitable fluorophore to a nucleotide can be carried out by conventional means. Suitably labelled nucleotides are also available from commercial sources. The label is attached in a way that permits removal, after the detection step. This may be carried out by any conventional method, including:

### I. Attacking the Signal Itself:

### a) Bleaching

i) Photobleaching
ii) Chemical bleaching

### a) Quenching of Fluorescence

i) By antibodies raised against the fluor (e.g. anti-fluorescein, anti-Oregon green)
ii) By FRET (the incorporation of a quencher next to a signal can be used to quench the signal, e.g. Taqman strategy)

### c) Cleavage of Signal

i) Chemical cleavage (e.g. reduction of a disulfide bridge between the base and the signal)
ii) Photocleavage (e.g. introduction of a nitrobenzyl or tert-butylketon group)
iii) Enzymatic (e.g. α-chymotryspin digestion of peptide linker)

### II. The Signal Bearing Nucleotide:

### c) Exonucleolytic Removal

i) 3′-5′ Exonucleolytic degradation of filled-in nucleotides (e.g. exonuclease III or by activating the 3′-5′ exonucleolytic activity of DNA polymerase when there is an absence of certain nucleotides)

### d) Restriction Enzyme Digestion

i) Digestion of double-stranded DNA bearing the signal (e.g. ApaI, DraI, SmaI sites which can be incorporated at the stop signals).

An alternative to the use of labels that permit removal, is to use inactivated labels that are reactivated during a biochemical process.

The preferred method is by photo or chemical cleavage.

When the label is a fluorophore, the fluorescent signal generated on incorporation may be measured by optical means, e.g. by a confocal microscope. Alternatively, a sensitive 2-D detector, such as a charge-coupled detector (CCD), can be used to visualise the individual signals generated.

The general set-up for optical detection is as follows:

| | |
|---|---|
| Microscope: | Epi-fluorescence |
| Objective: | Oil emersion (100X, 1.3 NA) |
| Light source: | Lasers or lamp |
| Filters: | Bandpass |
| Mirrors: | Dichroic mirror and dichroic wedge |
| Detectors: | Photomultiplier tubes (PMT) or CCD camera |

Variants may also be used, including:

### A. Total Internal Reflection Fluorescence Microscopy (TIRFM)

| | |
|---|---|
| Light source: | One or more lasers |
| Background control: | No pinhole required |
| Detection: | CCD camera (video and digital imaging systems) |

### B. Confocal Laser Scanning Microscopy (CLSM)

| | |
|---|---|
| Light source: | One or more lasers |
| Background reduction: | One or several pinhole apertures |
| Detection: | a) A single pinhole: Photomultiplier tube (PMT) |
| | detectors for different fluorescent wavelengths |
| | [The final image is built up point by point and |
| | over time by a computer]. |
| | b) Several thousand pinholes (spinning Nipkow |
| | disk): CCD camera detection of image [The final |
| | image can be directly recorded by the camera] |

### C. Two-Photon (TPLSM) and Multiphoton Laser Scanning Microscopy

| | |
|---|---|
| Light source: | One or more lasers |
| Background control: | No pinhole required |
| Detection: | CCD camera (video and digital imaging systems) |

The preferred methods are TIRFM and confocal microscopy.

According to the first aspect of the invention, the target polynucleotide is contacted with at least two probes that are complementary to non-overlapping, adjacent sequences of the target polynucleotide. As used herein, the term “adjacent” refers to two sequences that are adjoining in a single polynucleotide, with preferably no bases separating them. As ligation reactions can occur between different polynucleotides separated by 1 or 2 bases, this is also within the scope of the present invention and therefore “adjacent” should also be taken to include a 1 or 2 base separation. After the probes have been added and subjected to hybridising conditions, a ligation step is carried out. Conditions suitable for ligation of polynucleotides will be apparent to one skilled in the art. If at least two probes hybridise to adjacent sequences on the target polynucleotide, the ligation step will link these probes to form a single polynucleotide, by the formation of a phosphodiester bond between adjacent terminal nucleotides. This ligated product, which contains information on the target polynucleotide, may then be detected and characterised.

As a non-limiting, illustrative example of this aspect, two probes may be used. When both probes hybridise to the target, they are then ligated together to form a single molecule. This ligation step will only occur when both probes are hybridised. It will therefore be appreciated that if one, or both, of the oligonucleotide probes is not complementary to the target molecule, then hybridisation and ligation will not occur and a single molecule-comprising two probes will not be formed.

According to the simplest embodiment of this aspect, the method may be used to detect whether or not a target sequence is present in a sample. In a preferred embodiment, the sample contains a variety of DNA sequences, for example genomic DNA. At least two probes are used that are complementary to two adjacent sequences in the target molecule. If the target is present in the sample, the probes will hybridise, be ligated and a single probe molecule will be formed, which can then be detected. If the target is not present in the sample, a single ligated probe molecule will not be formed and will therefore not be detectable.

The presence of a mutation in the target polynucleotide may also be detected. The probes may be designed to be complementary to a mutant sequence or a wild-type sequence in the target. If the probes are designed to be complementary to the mutant sequence, the presence of a single ligated probe molecule after the ligation step indicates that the mutant sequence was present in the original sample, and its absence indicates that the target mutant sequence was not present in the original sample. Once a single ligated probe is produced, the sequence can be characterised by a read-out step. It will be clear to one skilled in the art that the reverse situation will work equally well, i.e. the probes are complementary to the wild-type sequence. In this case, the presence of a single ligated probe molecule after the ligation step indicates that the wild-type sequence was present in the original sample, and its absence indicates that the target wild-type sequence was not present in the original sample.

A combination of wild-type and mutant complementary probes may be used to identify if both wild-type and mutant sequences are present in the original sample. For example, one allele may contain the wild-type whilst a second allele contains the mutant sequence, both alleles being present in a genomic DNA sample.

A second aspect of the invention involves the identification of the order of at least two target sequences within a target polynucleotide. This may be used, for example, to investigate gross rearrangements or to determine the order of markers on a target sequence. The distance between the target sequences can also be determined. In a preferred embodiment, the target polynucleotide is contacted under stringent hybridising conditions, with at least two polynucleotide probes, both of which contain a second region encoding information on the sequence to which the probe hybridises. The probes do not have to hybridise to adjacent sequences on the target polynucleotide. In a preferred embodiment, the probes are complementary to non-adjacent regions of the target polynucleotide. Once the probes have been hybridised to the target polynucleotide, a polymerase reaction is carried out to incorporate nucleotides between the two probes, wherein one of the hybridised probes will act as a primer for polymerisation. Preferably, the polymerase reaction is quantitative, as previously described.

In order for the polymerase reaction only to fill in the nucleotides between the probes, and not to displace the probes themselves, a non-displacing polymerase must be used. Non-strand displacing polymerases are well known in the art, examples include but are not limited to Taq polymerase, E. coli DNA polymerase I and T4 DNA polymerase.

A ligation reaction is then performed to covalently link the probes to the polynucleotide created by the polymerase, to form a single polynucleotide containing the probes. The order of the target sequences, and the distance between them, can then be detected by characterising the single polynucleotide containing the probes, by detecting the order of and distance between the second regions of each probe.

The method of the invention is illustrated as follows:

A target DNA polymer within a genomic DNA sample is suspected to contain a SNP at a specific site. A first polynucleotide probe contains a first region complementary to the 10 bases immediately upstream of the SNP site but not including the SNP site, and a second region containing a series of 10 3-base units, each unit representing a single base in the first region. Each unit contains a base that can act as a stop-signal, a single-base spacer and a base suitable for identification in the read-out phase. The second region is upstream of the first region.

Four different second polynucleotide probes are used; each contains a first region of 10 bases that differ only at the base complementary to the SNP site, i.e. the first base is A, T, G or C in each of the four different second probes. The remaining 9 bases in each probe are identical, and are complementary to the 9 bases immediately downstream of the mutation site. These second polynucleotide probes also contain a second region containing a series of 10 3-base units, each unit representing a single base in the first region. In each second polynucleotide, the second region is downstream of the first region.

The genomic DNA sample is melted to provide a single-stranded target sequence and the first polynucleotide probe is added to the sample, together with each of the 4 second polynucleotide probes, under hybridising conditions. The first region of the first probe will hybridise to the 10 bases immediately upstream of the SNP site. The first region of one of the four second polynucleotides will hybridise with 100% complementarity to the SNP site and 9 bases downstream. The remaining 3 second polynucleotides, which are not complementary to the SNP site, will only hybridise to the 9 bases downstream of the SNP site. A ligation reaction is then carried out, by the addition of a ligase enzyme under conditions suitable for ligase activity. This ligation will only join the first polynucleotide probe to the second polynucleotide probe when the second probe is 100% complementary to the target sequence, i.e. it is complementary to the SNP. The remaining 3 second probes, that are not complementary at the SNP site, will not be ligated to the first probe. The ligation forms a single, ligated probe molecule that contains the first polynucleotide probe and the second polynucleotide probe that is complementary to the SNP site. The ligated probe contains the two first regions, flanked by the two second regions.

The single, ligated probe molecule is amplified in a polymerase reaction. The amplified polynucleotide contains sequence information, encoded in the second region of the first and second polynucleotide probes, on the 20-base region spanning the SNP, including the sequence at the SNP site. This sequence can be determined in any suitable read-out step.

1. A method for identifying the presence of a single-stranded target polynucleotide in a sample, comprising the steps of:
(I) contacting the target polynucleotide, under hybridising conditions, 5 with at least first and second polynucleotide probes, each of which comprise a
first region complementary to adjacent non-overlapping regions of the target polynucleotide;
(ii) ligating together those first and second polynucleotides that hybridise to the target polynucleotide;
(iii) optionally, amplifying any ligated polynucleotides; and
(iv) determining whether the target polynucleotide is present in the original sample, by detecting any ligated polynucleotide, wherein at least one of the first and second polynucleotides comprise a second region having a defined polynucleotide sequence, with each individual nucleotide of the first region being represented by at least two nucleotides on the second region, and the ligated polynucleotide being identified by determining the second region of at least one of the first and second polynucleotide probes.

2. A method according to claim 1, wherein the first and second polynucleotide probes are DNA.

3. A method according to claim 1, wherein the first and second polynucleotide probes are RNA.

4. A method according to claim 1, wherein each of the bases A, T(U), G and C of the first region is represented by a combination of two sequential units in the second region, with each base represented by a different combination of the two units.

5. A method according to claim 1, wherein the first and second polynucleotide probes are labelled with a fluorescent moiety.

6. A method according to claim 1, wherein step (ii) comprises the addition of a ligase enzyme under conditions suitable for ligase activity.

7. A method according to claim 1, wherein the ligated polynucleotide of step (ii) is amplified in a polymerase reaction.

8. A method according to claim 7, wherein the polymerase reaction is quantitative and the amount of amplified polynucleotide is monitored.

9. A method according to claim 1, wherein the target polynucleotide is RNA.

10. A method according to claim 1, wherein the target polynucleotide is mRNA.

11. A method according to claim 1, wherein the target polynucleotide is DNA.

12. A method according to claim 1, for detecting a target polynucleotide that differs by one or more nucleotides compared to the wild-10 type sequence.

13. A method according to claim 12, for detecting a deletion, insertion or substitution.

14. A method according to claim 1, for detecting a single nucleotide polymorphism.

15. A method according to claim 1, for detecting a wild-type target sequence.

16. A method according to claim 1, wherein step (i) comprises the binding of a plurality of the same or different first and second polynucleotide probes to the target polynucleotide.

17. A method for identifying the order of at least two target sequences on a target polynucleotide, comprising:
(i) contacting the target polynucleotide, under hybridising conditions, with at least a first and second polynucleotide probe, each of which comprise a first region complementary to non-adjacent regions of the target polynucleotide;
(ii) performing a polymerase reaction to incorporate nucleotides between the at least two probes to thereby form a third polynucleotide; and
(iii) determining the order of, and distance between, the two target sequences on the target polynucleotide, by detecting any third polynucleotide,
wherein the first and second polynucleotides comprise a second region having a defined polynucleotide sequence, with each individual nucleotide of the first region being represented by at least two nucleotides on the second region, and
the order of and distance between the probes in the third polynucleotide being identified by detecting the second region of the first and second polynucleotide probes.


SEQUENCE LISTING (4 sequences)

SEQ ID NO: 1 - DNA, 10 bp, Artificial sequence
  template sequence
ttttttaccc

SEQ ID NO: 2 - DNA, 10 bp, Artificial sequence
  template sequence
ttttttgccc

SEQ ID NO: 3 - DNA, 10 bp, Artificial sequence
  template sequence
ccccccattt

SEQ ID NO: 4 - DNA, 10 bp, Artificial sequence
  template sequence
ccccccgttt